(19) United States
(12) Patent Application Publication
(10) Pub. No.: US 2005/0123526 A1
(43) Pub. Date: Jun. 9, 2005

(54) Administration of growth factors for neurogenesis and gliagenesis

(75) Inventor: Lisa L. Shafer, Stillwater, MN (US)

Correspondence Address: MEDTRONIC, INC., 710 MEDTRONIC PARKWAY NE, MS-LC340, MINNEAPOLIS, MN, 55432-5604, US

(73) Assignee: MEDTRONIC INC., Minneapolis, MN (US)

(21) Appl. No.: 11/001,200

(22) Filed: Dec. 1, 2004

Related U.S. Application Data
(60) Provisional application No. 60/526,405, filed on Dec. 1, 2003.
(60) Provisional application No. 60/526,318, filed on Dec. 1, 2003.

(51) Int. Cl. A61K 48/00; A61N 1/18

(52) U.S. Cl. 424/93.21; 514/12; 607/47; 424/143.1

(57) ABSTRACT

Devices and methods for treating diseases associated with loss of neuronal function by cell replacement therapy are described. The methods are designed to promote proliferation, differentiation, migration, or integration of exogenous stem cells transplanted into the central nervous system (CNS). A therapy, such as an electrical signal or a stem cell enhancing agent, or a combination of therapies, is applied to a CNS region having damaged neuronal tissues, into which region exogenous stem cells are transplanted. A therapy may also be applied to a second region of the CNS to which neurons from the damaged CNS region are expected to project. The exogenous stem cells may be transfected with an electrically responsive genetic construct comprising an electrically responsive promoter and a target gene. Expression of the target gene, which may encode a gene product that promotes proliferation, differentiation, migration, or integration of the exogenous stem cell, may be closely controlled by application of an electrical signal.

### RELATED APPLICATIONS

This application claims the benefit of priority from provisional applications Ser. Nos. 60/526,405 and 60/526,318, both filed on Dec. 1, 2003, which provisional applications are each incorporated by reference herein in their respective entireties.

### BACKGROUND

Over the past several decades, the concept of neurological tissue grafting or exogenous stem cell transplantation has been investigated for its potential to treat neurodegenerative disease such as Parkinson's disease. Stem cell technology continues to evolve rapidly. Current approaches have resulted in some therapeutic successes but the establishment of long-term functional replacement is debatable and variable. In many cases it appears that the transplanted cells do not form or maintain the fully functional contacts essential for cell survival. The transplanted cell may fail at any given step in the pathway to becoming a functional replacement cell namely: proliferation, differentiation, migration or integration.

Some researchers have attempted to drive the differentiation of stem cells in vitro using various growth factors and differentiation factors prior to implanting the cells. Others have attempted to drive the differentiation of stem cells in vivo, after they have been implanted. However, these methods have generated very little in the way of therapeutic successes to date. In order to study the mechanisms involved in stem cell proliferation, differentiation, migration and integration, researchers can transfect exogenous stem cells in vitro with gene sequences thought to be involved in these processes. Using transfected exogenous stem cells to determine gene function has served as a valuable research tool but has not been applied as a therapeutic strategy to the same degree. Research to date suggests that several growth and differentiation factors may be involved in these processes and the particular agent or mix of several depends on the type of cell desired.

Examples of factors that encourage proliferation/expansion Interleukin-3 (IL-3), stem cell factor (SCF), and Flt-3 ligand (FL), Platelet-derived growth factor (PDGF), and epidermal growth factor(EGF), fibroblast growth factor-2 (FGF-2). A cocktail of several may be applied. For example, neuronal precursor cells have been expanded in the presence of both EGF and FGF-2. A specific example is provided by Lazzari et al. (2001) wherein, the highest expansion of cord blood HSC was obtained with a cocktail containing FL, thrombopoeietin (Tpo), IL-6 and IL-11.

Transcription factors such as Pax6 and Emx2 may be required for proliferation and patterning during neuronal development. Sonic hedgehog (SHH) is well known for its control of numerous processes during development as well as acting as a mitogen for embryonic neural stem cells. SHH may induce proliferation of adult stem cells. In the adult CNS, actions of BMP and noggin are believed to regulate the balance between neurons and astrocytes. Such gene sequences may be incorporated in exogenous stem cells via transfection prior to implantation.

Further, transforming growth factor-beta (TGF-b) family members have been demonstrated to have differentiation effects on ES cells (Schuldiner M. 2000) and neural crest stem cell differentiation (Shah N. M. (1996); White P. M. (2001)). Other agents that contribute to differentiation and that may be administered to optimize the microenvironment are Wnt factors, integrins, and extracellular matrix components. A mix of factors may be applied to differentiate a group of stem cells into a particular type of neuron, after the cells were first encouraged to proliferate: For example, FGF-2, ascorbic acid, sonic hedgehog (SHH) and FGF-8 have been used to differentiate mouse ES cells to obtain dopaminergic and serotonergic neurons (Lee S. M. 2000).

Although extensive research continues in the areas of in vitro transfection of exogenous stem cells, very little has been reported on methods to control and regulate these exogenous transplanted cells, and in particular the expression of transfected elements in vivo. Researchers have taken advantage of inherent DNA sequences found upstream of a gene, which regulate the expression of the gene under different physiological conditions. To this end, recombinant elements have been developed to effectively introduce and express genes in many cell types. Several protocols have been published which have focused on pharmacologically-based control of gene expression. Generally the basis of these methods relies on the presence of a pharmacological agent to control the activation of the DNA promoter sequences. An example of this is the Tet-On/Tet-Off gene expression system, which is commercially available. The presence or absence of tetracycline or doxycycline will activate the promoter responsible for turning on gene expression. Administration of the activating pharmacological agent is generally done systemically in an effort to deliver the affecting transcription to the site of the action. Although technically effective at inducing gene expression, the possibility exists that systemic administration of pharmacological agents in vivo can result in unwanted side effects or toxicity in surrounding tissues. Further, because pharmacological agents reside in the body over a period of time, often for days, regulation of the gene promoter sequence is not tightly coupled from the time the activating agent is given until it is eliminated from the body.

WO 02/49669 discloses the controlled delivery of therapeutic gene products regulated in a patient via an electrical device. In WO 02/49669, an electrical pulse generator, e.g., a pacemaker, is used to closely modulate the time, frequency, and delivery amount of a given therapeutic product and to closely define the locus of delivery, such that tissues containing genetically engineered cells that have received electrically responsive promoter elements direct the expression of a therapeutic product upon receiving electrical stimulation. A system described in WO 02/49669 utilizes an electrical stimulus (provided by an electrical pulse generator) as a means to control the expression of electrically responsive promoters (ERPs) that have been transplanted or incorporated into the tissue of a mammal. The target gene of interest is operably linked to an electrically responsive promoter sequence to provide controlled expression by the ability to closely regulate the electrical stimulus. The ERP gene constructs can be delivered by standard gene transfection methods to cells grown in culture and then implanted into the patient, or delivered directly to tissues or cells in vivo through the use of an appropriate gene delivery vector (viral or non-viral). Implantable electrodes operably coupled to the pulse generator can then be used to electrically stimulate at a defined locus the electrically responsive promoters in transfected or transplanted cells, which consequently results in the controlled expression of operably linked DNA sequences.

The present disclosure relates to the use of exogenous stem cells, whether or not transfected with an ERP gene construct, as cell replacement therapy for CNS disorders. Such use of exogenous stem cells coupled with application of an electrical signal and a stem cell enhancing agent configured to promote proliferation, differentiation, migration, or integration of the exogenous stem cell has not been described. Additionally, the further application of a therapy to a CNS location to which transplanted exogenous stem cells or neuronal derivatives thereof are predicted to project have not been described.

### SUMMARY

The present disclosure describes improved devices and methods configured for using exogenous stem cells, whether or not transfected with an ERP gene construct, as cell replacement therapy for CNS disorders. This disclosure describes the combination of electrical and chemical therapies to optimize the proliferation, differentiation, migration, or integration of exogenous stem cells. In addition, this disclosure describes administration of stem cell enhancing agents or electrical signals at more than one location to enhance treatment of disorders associated with loss of neuronal function.

In an embodiment, the invention provides a therapy delivery system. The therapy delivery system comprises a housing. An electrical signal generator, such as a pulse generator, and a pump are disposed within the housing. A reservoir is operably coupled to the pump and contains one or more stem cell enhancing agents, such as a growth factor. The system further comprises genetically engineered stem cells comprising a target gene operably coupled to an electrically responsive promoter. The target gene may encode a gene product that promotes the proliferation, differentiation, migration, or integration of the genetically altered stem cells. The cells are operably coupled with the electrical signal generator.

An embodiment of the invention provides a method for treating a disease associated with loss of neuronal function in a subject in need thereof. The method comprises transplanting an exogenous stem cell to a first CNS region that comprises damaged neuronal tissue. The exogenous stem cell may comprise an electrically responsive nucleic acid construct. The construct comprises an electrically responsive promoter and a target gene encoding a gene product capable of promoting the proliferation, differentiation, migration, or proliferation of the exogenous stem cell. The method further comprises implanting a lead in the subject such that an electrode of the lead is positioned in the first CNS region. An electrical signal is applied via the electrode to the first CNS region. The electrical signal is configured to promote proliferation, differentiation, migration, or integration of the exogenous stem cell. The promotion of proliferation, differentiation, migration, or integration may occur by applying an electrical signal configured to induce expression of the target gene product. The method further comprises implanting a catheter in the subject such that a delivery region of the catheter is positioned in the first CNS region. A first stem cell enhancing agent is applied to the first CNS region via the delivery region. The stem cell enhancing agent is capable of promoting proliferation, differentiation, migration, or integration of the exogenous stem cell. The method may further comprise implanting a therapy delivery element comprising a therapy delivery region in the subject and positioning the therapy delivery region of the therapy delivery element in a second CNS region to which neurons from the first CNS region are predicted to project. A therapy may be applied to the second CNS region via the therapy delivery region to promote projections of the neurons from the first CNS region to the second CNS region. The method may further comprise intraventricularly or intrathecally delivering a second stem cell enhancing agent. The second stem cell enhancing agent may be the same or different than the first stem cell enhancing agent.

In an embodiment, the invention provides a method for treating a disease associated with loss of neuronal function in a subject in need thereof. The method comprises transplanting an exogenous stem cell to a first CNS region that comprises damaged neuronal tissue. The exogenous stem cell may comprise an electrically responsive nucleic acid construct that comprises an electrically responsive promoter and a target gene encoding a gene product capable of promoting the proliferation, differentiation, migration, or proliferation of the exogenous stem cell. The method further comprises implanting a first therapy delivery element, such as a lead or catheter, comprising a therapy delivery region, such as an electrode or infusion section, in the subject and positioning the therapy delivery region of the first therapy delivery element in the first CNS region. A first therapy is applied to the first CNS region via the therapy delivery region of the first therapy delivery element to promote proliferation, differentiation, migration, or integration of the exogenous stem cell. The first therapy may comprise an electrical signal that is capable of inducing expression of the target gene product. The method further comprises implanting a second therapy delivery element comprising a therapy delivery region in the subject and positioning the therapy delivery region of the second therapy delivery element in a second CNS region to which neurons from the first CNS region are predicted to project. A second therapy is applied to the second CNS region via the therapy delivery region of the second therapy delivery element to promote projections of the neurons from the first CNS region to the second CNS region. The first and second therapy are the same or different. The method may further comprise intraventricularly or intrathecally delivering a stem cell enhancing agent to promote the proliferation, differentiation, migration, or integration of the exogenous stem cell or a cell derived therefrom.

One or more embodiments of the invention provide advantages over existing devices and methods for treating diseases associated with diminished neuronal function. For example, the combined use of electrical signals and stem cell enhancing agents should prove more efficacious than either alone for cell replacement therapy and treatment of diseases associated with loss of neuronal function. The combination of electrical signals and soluble chemical agents should enhance the proliferation, migration, differentiation, or integration of exogenous stem cells. The deficiencies of application of only electrical or only chemical therapies at only one location may be overcome using the description provided herein. In addition, the use of electrically responsive nucleic acid constructs comprising target genes encoding for products that are capable of proliferation, migration, differentiation, or integration of the exogenous stem cells should prove to further enhance the therapy. The addition of additional therapy at CNS locations to which neurons from damaged tissue, in which exogenous stem cells are transplanted, project may serve as yet another advantage over existing therapies. These and other advantages will become apparent to one of skill in the art upon reading the disclosure presented herein.

### BRIEF DESCRIPTION OF THE DRAWINGS

FIG. 1 is a drawing of a therapy delivery system adapted to deliver therapy to a subject's brain.

FIG. 2 is a drawing of an implantable therapy delivery system adapted to deliver therapy to a subject's brain.

FIG. 3 is a drawing of a pulse generator therapy system.

FIG. 4 is a drawing of a pump system for delivering a therapeutic agent.

FIG. 5 is an illustration of therapeutic elements adapted to deliver therapy to two different brain regions, one region being a region containing damaged nervous tissue into which an exogenous stem cell is transplanted, the other representing a region to which neurons from the damaged region are predicted to project.

FIGS. 6-12 are flow charts illustrating various methods for treating a disease associated with loss of neuronal function.

FIG. 13 is a drawing of a therapy delivery device coupled to a therapy delivery element.

FIG. 14 is a drawing of a therapy delivery device coupled to two therapy delivery elements.

FIG. 15 is a drawing of a therapy delivery device having two therapy delivery units, each coupled to a therapy delivery element.

The drawings are not necessarily to scale.

### DETAILED DESCRIPTION

In the following descriptions, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense.

Definitions

All scientific and technical terms used in this application have meanings commonly used in the art unless otherwise specified. The definitions provided herein are to facilitate understanding of certain terms used frequently herein and are not meant to limit the scope of the present disclosure.

As used herein, “subject” means a living being having a nervous system, to which living being a device or method of this disclosure is applied. “Subject” includes mammals such as mice, rats, pigs, cats, dogs, horses, non-human primates and humans. A subject may be suffering from or at risk of a disease or condition.

As used herein, the terms “treat”, “therapy”, and the like are meant to include methods to alleviate, slow the progression, prevent, attenuate, or cure the treated disease.

As used herein, “disease associated with loss of neuronal function” means a disease, disorder, condition, and the like resulting from impairment of nervous tissue function. The impairment may result from damage to nervous tissue, such as a neuron or glial cell. Nervous tissue may be damaged genetically or through infection, disease, trauma, and the like. As used herein, “repairing damaged neural tissue” means improving, restoring, replacing the function of a damaged neuron. A neuron may be damaged genetically or through infection, disease, trauma, and the like.

As used herein, “promoting neurogenesis” refers to a series of events (including proliferation of a neural precursor or stem cell) that results in the appearance of a new neuron.

As used herein, “exogenous stem cell” means stem cells that are transplanted into a subject. Exogenous stem cells include multipotent, totipotent, pluripotent stem cells that are present in an organ or tissue of a subject. Such cells are capable of giving rise to a fully differentiated or mature cell types. A stem cell may be a bone marrow-derived stem cell, autologous or otherwise, a neuronal stem cell, an embryonic stem cell. A stem cell may be nestin positive. A stem cell may be a hematopoeietic stem cell. A stem cell may be a multi-lineage cell derived from epithelial and adipose tissues, umbilical cord blood, liver, brain or other organ.

The term “genetically engineered cell(s)” means cells that have had defined segments of nucleic acid purposefully introduced into the cell. The term “genetically engineered cell” is not meant to be limited by the means of introduction of the nucleic acid unless specifically so indicated.

The term “operably linked”, as used herein, denotes a relationship between a regulatory region (typically a promoter element, but may include an enhancer element) and the coding region of a gene, whereby the transcription of the coding region is under the control of the regulatory region. As used herein, “operably linked” refers to a juxtaposition of transcriptional regulatory elements such that the transcriptional function of the linked components can be performed. Thus, an ERP promoter sequence “operably linked” to a coding sequence refers to a configuration wherein the promoter sequence promotes expression (or inhibits the expression if a negative regulatory element) of the gene sequence upon electrical stimulation.

“Operably coupled”, in the context of a electrical signal generator and a tissue, refers to the transference of an electrical stimulus by an electrical signal generator to a tissue. A signal generator operably coupled with genetically engineered cells as described herein refers to a configuration where an electrical stimulus is delivered to the tissue area containing genetically engineered cells to cause expression of an operably linked target gene. Usually the signal is delivered from the signal generator through leads to electrodes in contact with the tissue.

An “electrically responsive promoter” or “ERP” is a promoter that contains a genetically engineered electrically responsive element that modulates transcription of an operably linked target gene in a cell upon the delivery of an electrical stimulus. Modulated transcription may be positive or negative, and may change the relative transcriptional amount over time by an amount that is equal to or approximately 2, 4, 6, 10, 20, 50, 100, or 1000 fold or greater than unstimulated cells over 1, 2, 4, 8, 16, 24, 48, or 72 hours. In one embodiment the ERP promoter is an any promoter. The term “promoter” refers to a nucleic acid sequence that directs transcription, for example, of DNA to RNA. As referred to herein the promoter includes the 5′ flanking sequences that promote transcription. A promoter may contain several regulatory sequences. A constitutive promoter generally operates at a constant level and is not regulatable. The ERP promoters of the discussed herein can be induced by electrical signals.

As used herein, “electrically responsive nucleic acid construct” refers to a nucleic acid sequence comprising an electrically responsive promoter operably linked to a target gene such that the target gene can be expressed upon delivery of an appropriate electrical signal to the cell.

Delivery of Therapy

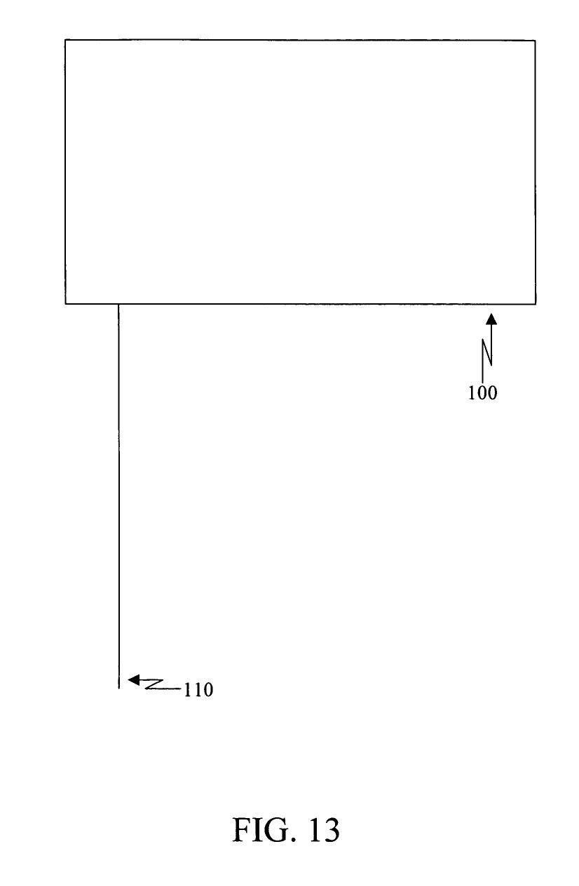

Referring to FIG. 13, a therapy delivery device 100 may be operably coupled to therapy delivery element 110. A therapy delivery region (not shown) of therapy delivery element 110 may be positioned in a subject's centeral nervous system (CNS) to deliver a therapy. The therapy may be a therapeutic agent or an electrical signal. Therapy delivery element 110 may be a catheter or a lead, and therapy delivery region may be an infusion region of a catheter or an electrode.

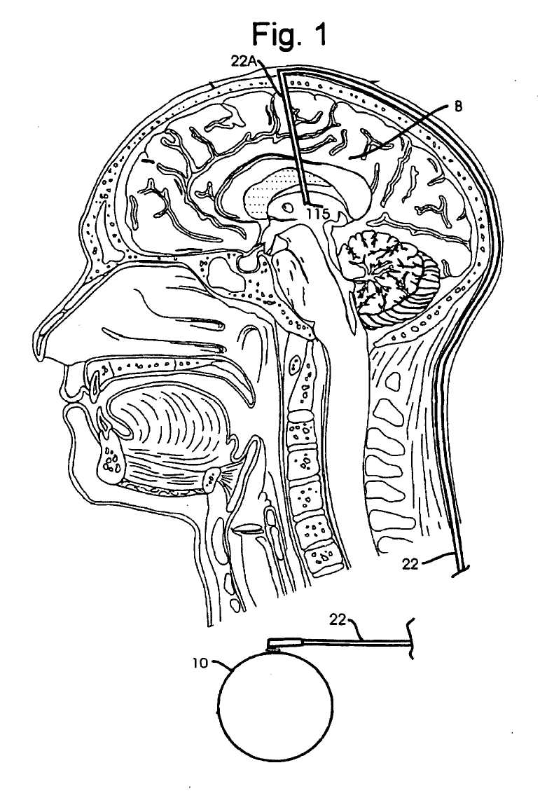

Referring to FIG. 1, a therapy delivery region 115 of a therapy delivery element 22 may be positioned to deliver therapy within a brain region of a subject. Therapy delivery region 115 is shown at distal portion of therapy delivery element 22, but it will be understood that therapy delivery region 115 may be located at any position along therapeutic element 22. The therapy delivery element 22 may be coupled to a therapy delivery device 10. The device 10 may be, e.g., a signal generator or a pump for delivering a therapeutic agent. The device 10 may be implantable. There may be more than one therapy delivery element 22 coupled to the device 10. An individual delivery element 22 may be divided into two portions 22A and 22B that may be implanted into the brain bilaterally. Alternatively, a second device 10 and therapeutic element may be used to deliver therapy to a corresponding brain region in a second brain hemisphere.

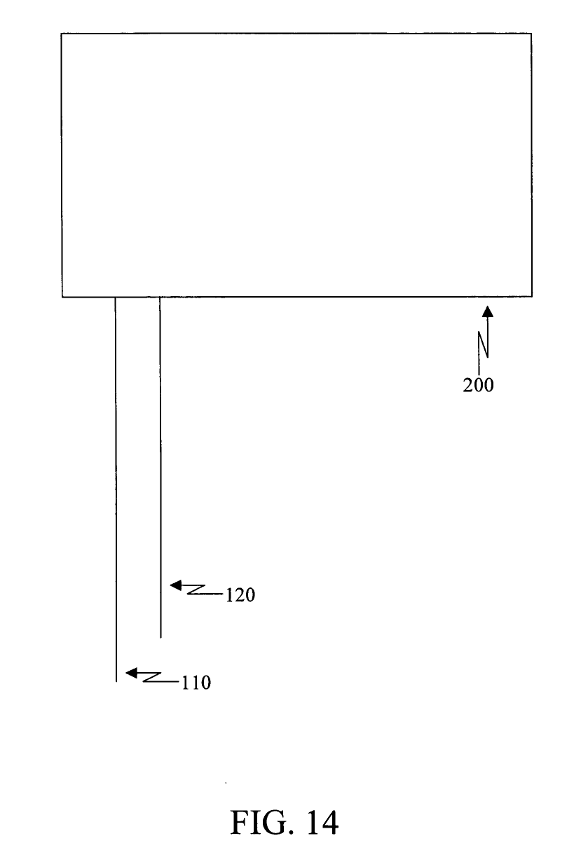

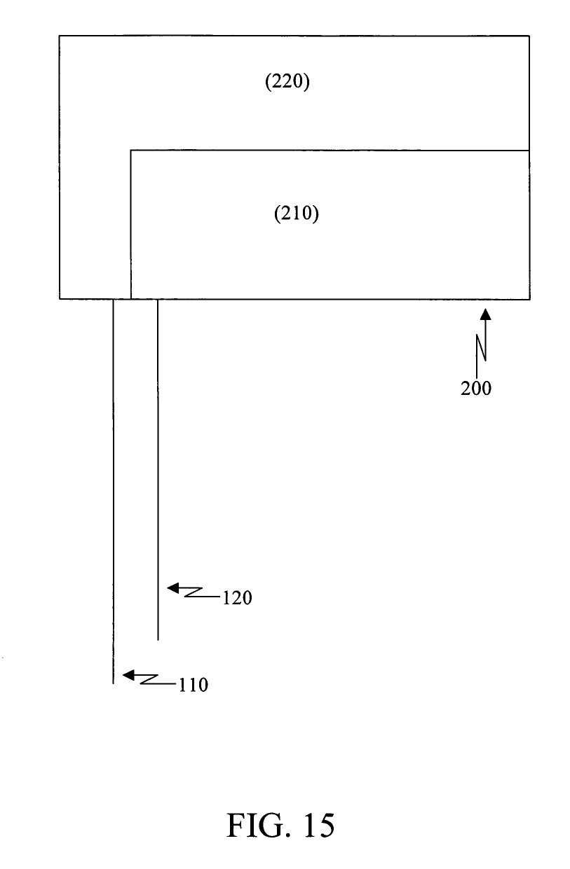

Referring to FIG. 14, therapy delivery device 200,100 operably coupable to two therapy delivery elements 110, 120, 22 is shown. It will be understood that therapy delivery device 200,100 may be coupled to more than two therapy delivery elements 110, 120, 22. As shown in FIG. 15, therapy delivery device 200,100 may have two therapy delivery units 210, 220, which may be the same or different. For example, therapy delivery units 210, 220 may both comprise electrical signal generators, may both comprise pump mechanisms, or one may comprise a signal generator and one may comprise a pump mechanism. Devices 200, 100 comprising a combination of a electrical signal generator and an a pumping mechanism may take the form of a device described in, e.g., U.S. Pat. No. 5,119,832, U.S. Pat. No. 5,423,877 or U.S. Pat. No. 5,458,631, each of which are hereby incorporated herein by reference in their entireties. It will be understood that device 200, 100 may have more than two delivery units 210, 220.

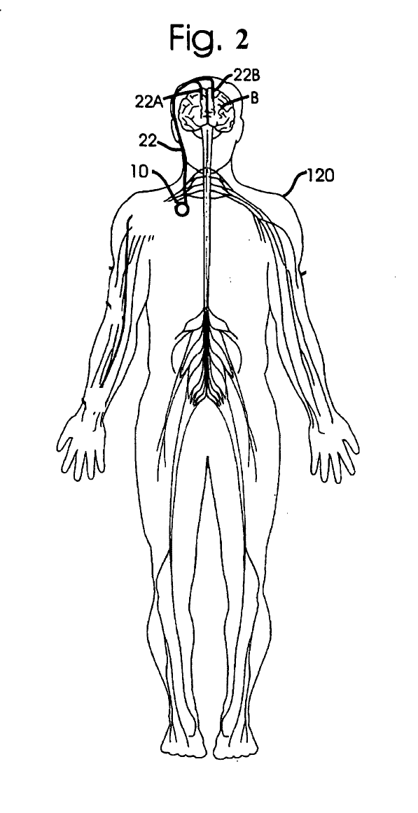

Referring to FIG. 2, device 200, 100, 10 may be implanted in a human body 120. The device 200, 100, 10 may be implanted in the location shown or any other location suitable for the coupled therapeutic element 120, 110, 22 to deliver therapy to a region of the CNS. Such other suitable locations include the abdomen, the cranium, and the neck. Therapy delivery element 120, 110, 22 may be divided into twin portions 22A and 22B that are implanted into the brain bilaterally. Alternatively, portion 22B may be supplied with therapy from a separate element 120, 110, 22) and device 200, 100, 10.

Electrical Signal

In an embodiment of the invention, an electrical signal is applied to a region of a subject's CNS. The CNS region may be, e.g., a brain region in which an exogenous stem cell is transplanted, a brain region containing damaged nervous tissue, a brain region neurons from a brain region containing damaged nervous tissue are predicted to send projections, and the like. An “electrical signal” refers to an electrical or electromagnetic signal. In an embodiment, the signal has a pulse width, a frequency, an amplitude, a polarization, and a duration. An electrical signal may be depolarizing, may be hyperpolarizing, may increase the likelihood that a neuron will undergo an action potential, or may decrease the likelihood that a neuron will undergo an action potential. For example, a depolarizing signal may be a threshold or subthreshold (i.e., not sufficient to cause a neuron to undergo an action potential) signal. An electrical signal may be produced by any means suitable for application of the signal to a region of the subject's CNS. In an embodiment, the electrical signal is generated by a pulse generator. The pulse generator may be implantable.

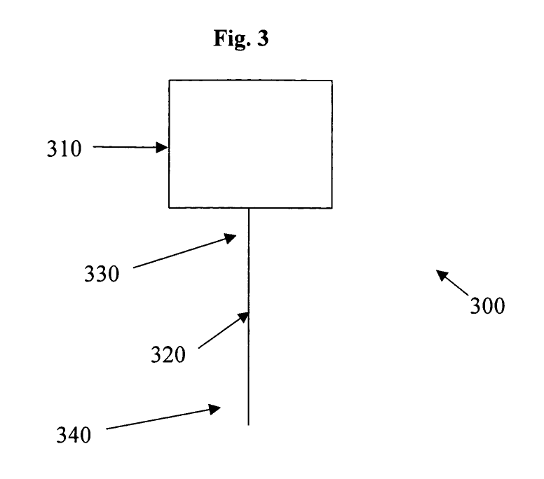

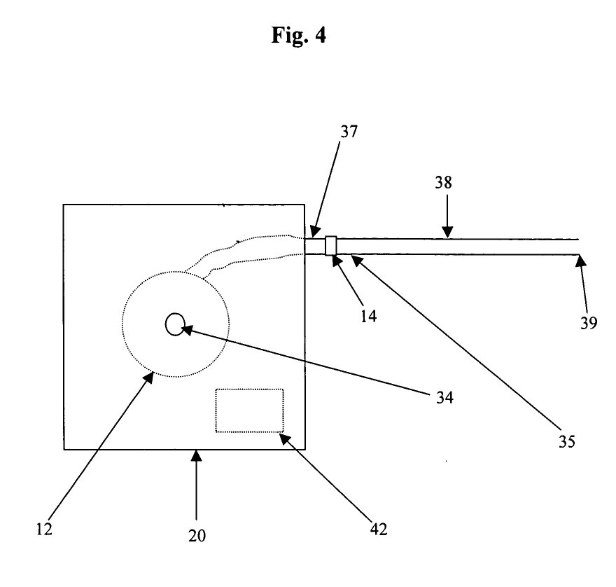

Referring to FIG. 3, a pulse generator system 300 includes a pulse generator 310 and one or more leads 320. Any suitable pulse generator 310 and lead 320 may be used in accordance with various embodiments of the invention. A suitable pulse generator 310 includes Medtronic Model 3625 test stimulator. A suitable lead 320 includes any of the Medtronic leads sold with the Model 3625, such as Model YY005093 IR or other custom made leads. Lead 320 is electrically coupled to pulse generator 310. A proximal portion 330 of the lead 320 is coupled to the pulse generator. A distal portion 340 of the lead 320 may be positioned to apply an electrical signal produced by a pulse generator 310 to a brain region into which an exogenous stem cell is transplanted.

The pulse generator 310 may be implantable as shown for the device 10 in FIG. 2. An implantable pulse generator system 300 includes an implantable pulse generator 310, such as Medtronic's Model 7425 Itrel or Model 7427 Synergy. Typically, the implantable pulse generator 310 will be electrically coupled to one or more leads 320. Suitable leads 320 are known and can typically be purchased with implantable pulse generators 310. Examples of suitable leads 320 include Medtronic's Pisces leads, Resume leads and other custom builds. The one or more leads 320 may be positioned to apply an electrical signal produced by the implantable pulse generator 310 to a brain region into which an exogenous stem cell is transplanted.

A pulse generator 310, whether or not it is implantable, may be programmed to adjust electrical signal parameters such as pulse width, frequency, amplitude, polarization, and duration. A physician or other person skilled in the biomedical arts with respect to neurostimulation will understand that the parameters may be optimized to achieve an electrical signal having desired properties. The parameters and the location of the application of the electrical signal may be varied to optimize therapeutic effect. In an embodiment, an electrical signal having a voltage of between about 1 mV to about 10 mV, a frequency of about 1 Hz to about 1000 Hz, and a pulse width of about 1 μsec to about 500 μsec is applied to a CNS region to promote the proliferation, differentiation, migration or integration of a stem cell.

Delivery of Therapeutic Agent

In an embodiment of the invention, one or more stem cell enhancing agents may be administered to a CNS region of a subject. The CNS region may be, e.g., a brain region in which an exogenous stem cell is transplanted, a brain region containing damaged nervous tissue, a brain region neurons from a brain region containing damaged nervous tissue are predicted to send projections, and the like. It will be understood that therapeutic agents in addition to stem cell enhancing agents may also be administered. The additional therapeutic agents may be beneficial for treating a disease associated with loss of neuronal function.

Referring to FIG. 2, a system for delivering a therapeutic agent to a brain region of a subject is shown. The device 20 comprises a pump 40, a reservoir 12 for housing a composition comprising a therapeutic agent, such as a growth factor, and a catheter 38 having a proximal portion 35 operably coupled to the pump 40 and an infusion section 39 adapted for infusing the composition to the brain region of the subject. The device 20 may be an implantable pump, as shown regarding the device 10 in FIG. 2, or may be an external pump. The device 20 may have a port 34 into which a hypodermic needle can be inserted to inject a quantity of therapeutic agent into reservoir 12. The device 20 may have a catheter port 37, to which the proximal portion 35 of catheter 38 may be coupled. The catheter port 37 may be operably coupled to reservoir 12. A connector 14 may be used to couple the catheter 38 to the catheter port 37 of the device 20. The device 20 may contain a microprocessor 42 or similar device that can be programmed to control the amount of fluid delivery. The device may take the form of Medtronic's SynchroMed EL or SynchroMed II infusion pump system.

It will be understood that a therapeutic agent may be administered to a brain region without use of a pump system 20.

Stem Cell Enhancing Agent

In an embodiment of the invention, one or more stem cell enhancing agent may be administered in addition to a stimulation signal. As used herein, a “stem cell enhancing agent” is an agent that alone or in combination with another stem cell enhancing agent or an electrical signal increases the likelihood that a stem cell will migrate, proliferate, differentiate, integrate or release a factor that may result in a neural cell migrating, proliferating, differentiating, or integrating. Stem cell enhancing agents are chemical compounds and may be small molecule chemical agents; nucleic acids; including vectors, small inhibitory RNA, ribozymes, and antisense molecules; polypeptides, and the like. While some stem cell enhancing agents may affect the ability of a cell to selectively proliferate, differentiate, migrate, or integrate, it will be understood that many stem cell enhancing agents will affect the ability of a cell to undergo a combination of more than one of proliferate, differentiate, migrate and integrate. Accordingly, a discussion of a stem cell enhancing agent as an agent that, e.g., promotes proliferation does not necessarily indicate that the agent may not also promote one or more of differentiation, migration, and integration. It will also be understood that a stem cell enhancing agent may differentially affect proliferation, differentiation, migration, and integration based upon the location in which the agent is administered.

A stem cell enhancing agent may be a growth factor. Any growth factor capable of repairing damaged neural tissue and/or promoting neurogenesis may be administered. Exemplary suitable growth factors include glial-derived neurotrophic factor (GDNF), brain-derived neurotrophic factor (BDNF), fibroblast growth factor (FGF), vascular endothelial growth factor (VEGF), nerve growth factor (NGF), neurotrophin (NT), transforming growth factor (TGF), ciliary neurotrophic factor (CNTF), epidermal growth factor (EGF), insulin-like growth factor (IGF), stromal cell factor (SCF), notch, heparan sulfate proteoglycans (HSPGs) and growth factors within these classes such as, for example, NT-3, IGF-1, FGF-2, SCF-1 and TGF-alpha. More than one growth factor may be administered. Each growth factor may be administered in the same brain region or may be administered in different locations. Any amount of a growth factor may be administered. Preferably, an amount of a growth factor capable of promoting stem cell proliferation, differentiation, migration, or integration, when administered alone or in combination with stimulation and/or additional therapeutic agents, is administered. It will be understood that that the efficacy of a growth factor may be enhanced by a cofactor. For example, administration of cofactor cystatin C and IGF may enhance the efficacy of FGF-2. In an embodiment of the invention, daily doses of growth factors administered are in the range of about 0.5 micrograms to about 100 micrograms. For specific daily dose ranges for NGF, BDNF, NT-3, CNTF, IGF-1, and GDNF that may be administered, see U.S. Pat. No. 6,042,579, which is incorporated herein by reference in its entirety.

Any growth factor may be administered. Some growth factors may be referred to in the art as mitogens. In addition to the growth factors listed above, other mitogens suitable for use in accordance with the teachings of this disclosure include bone morphogenic proteins (BMP), noggin, erythropoietin, and leukemia inhibitory factor (LIF).

A growth factor or other stem cell enhancing agent may be a chemoattractant agent. A chemoattractant agent is an agent that directs a migrating cell to a particular region or an agent that directs neuronal projections to a particular agent. Examples of chemoattractant agents include stromal-cell-derived factor (SCF-1), fractalkine, growth related oncogene alpha (GRO-α), IL-8, MIP-1a, MIP-1b, MCP-1, MCP-2, MCP-3, GRO-a, GRO-b, GRO-g, RANTES, and eotaxin

A stem cell enhancing agent may be an agent that inhibits factors that prevent extensive cell replacement. Such agents include an anti-nogo antibody, a p75ntr antagonist, a Rho-kinase inhibitor, and a nogo-66 receptor antagonist.

A stem cell enhancing agent may include agents that increase the likelihood that a neuron will undergo an action potential. Such agents include glutamate receptor agonists, such as LY 354740 or 5-dihydroxyphenylglycine (DHPG) and GABA receptor antagonists, such as CGP56433A or bicuculline.

Other neurotransmitters and agonists of their receptors that may be useful for promoting the proliferation, differentiation, migration, or integration of a stem cell include norepinepherine, acetylcholine, dopamine, serotonin, and the like.

In an embodiment, a stem cell enhancing agent is a transcription factor. Exemplary transcription factor include Pax6, EMX2, SHH, a member of the NeuroD family, a member of the CREB family, c-fos, myocyte enhancer factor-2 (MEF-2) and basic helix-loop-helix (bHLH) transcription factors.

Exogenous Stem Cells

In an embodiment of the invention, an exogenous stem cell is transplanted in the CNS of a subject at a location comprising damaged nervous tissue. Any exogenous stem cell capable of forming a mature nervous cell, such as a neuron or a glial cell, may be transplanted into the subject. Exogenous stem cells may be isolated using any known or future developed technique. For example, a stem cell may be isolated from an embryo, from a tissue or from an organ, including skin, and may be considered an embryonic stem cell or an adult stem cell. Conversely, an established stem cell line may be used.

Transplanted cells or grafts may be derived from auto-, alla- or xeno- graphic sources. Transplanted or grafted cells for brain tissue can be chosen from the group consisting of: adult fibroblasts, fetal fibroblasts, adult smooth muscle cells, fetal smooth muscle cells, endothelial cells, and skeletal myoblasts, embryonic cells, cord blood cells, adult stem cells of any organ such as brain, liver, heart, or bone marrow. Procedures for isolation of these cell types are known in the field and described elsewhere.

Exogenous stem cells may be introduced into a region of a subject's CNS comprising damaged nervous tissue using any known or future developed method. For example, exogenous stem cells may be introduced by direct injection, injection through a catheter, and the like.

An exogenous stem cell introduced into a region of a subject's CNS may or may not comprise an electrically responsive nucleic acid construct.

Ex Vivo Construction of ERP Stem Cells

In an embodiment, an exogenous stem cell comprising an electrically responsive nucleic acid construct is introduced into a region of a subject's CNS containing damaged nervous tissue. The electrically responsive nucleic acid construct comprises an electrically responsive promoter (ERP) and a target gene. The target gene may encode a gene product that promotes the proliferation, differentiation, migration, or integration of a stem cell. For example, the target gene may encode CNTF, GDNF, BDNF, FGF, VEGF, NGF, TNB, NT-3, TGF-alpha, TGF-beta, EGF, IGF-1, NT-4, NT-5, EGF, CNTF, SCF, c-fos, NeuroD2, pax6, emx2, SHH, noggin, IL-3 FL, PDGF, FL, Tpo, IL-6 IL-11, or an active derivative or fragment thereof. The nucleic acid construct may comprise more than one target gene. Alternatively, more than one nucleic acid construct may be introduced into an exogenous stem cell.

Nucleic acid constructs comprising ERPs can be introduced into stem cells ex vivo in any known or future developed manner. For example, such constructs may be introduced as described in WO 02/49669, which patent application is incorporated herein by reference in its entirety. In WO02/49669, Schu et al. have demonstrated that ERPs can be introduced into primary and secondary cells of mammalian origin and that ERP promoters can be stably integrated and operably linked to an exogenous genes using a wide variety of vectors.

The generation of different specialized cell types of the mammalian organism requires the establishment of diverse gene expression patterns that characterize the individual cell types. These patterns are formed through the combinatorial action of transcriptional regulatory proteins, some of which have the capacity to direct multipotent stem cells to assume a specific developmental fate. For example PU.1 which commits multipotent hematopoeietic cells to the myeloid lineage and C/EBPα which can instruct progenitor cells to differentiate into adipocytes, neutrophil granulocytes and eosinophils (Nerlov, C. and Grav, T. (1998); Nerlov, C et al., 1998).

In an embodiment, neurologic factors are produced from neural cells. Neural cells may be transfected in vivo or ex-vivo with the relevant gene under control of an electrically responsive promoter. Where neural cells are transfected ex-vivo they are then transplanted into the desired site in the neural tissue. Within the range of transplanted neural cells, include mature neuronal cells, glial cells (e.g., astrocytes, oligodendrocytes), as well as neural stem cells and the like.

An advantage to the use of ERP transfected primary or cultured cells of the present invention is that the number of cells required may be reduced and location of their delivery can be specified. Further, the proliferation, differentiation, migration and integration of the exogenous cell may be controlled by the location of electrodes and the period of electrical stimulation. Additionally, the exogenous cells may be controlled by the location of the catheter delivering a therapeutic agent to encourage the proliferation, differentiation, migration and integration of said cells.

In its simplest mode, to stimulate the electrically responsive elements within the cells of a patient, one would simply turn on the electrical signal generating device. Programming would be desired to be sure the amplitude of the electrical stimulation was sufficient to be turning on the gene. The appropriate amplitude would be determined as the lowest amplitude or 2×, 3×, 4× or 5× the lowest amplitude, or as the case may be that elicits a therapeutic outcome. In the absence of a detectable therapeutic result, a pacing amplitude may be set using an assay for the generated protein, or empirically using in vitro data on the amplitude versus distance from the cell to affect stimulation.

One or more electrically responsive construct may be transfected into an exogenous cell to be transplanted into a CNS region comprising damaged nervous tissue. Alternatively, different cells may be transfected with different constructs. The different constructs may contain ERPs that can be turned on when subjected to electrical signals comprising different parameters. Any known or future discovered or developed ERPs sensitive to various stimulation parameters may be used.

Brain Region with Damaged Tissue

In an embodiment of the invention, an electrical signal or a stem cell enhancing agent may be applied to a region of a brain having damaged neural tissue or damage to the glial cells. A therapy (i.e., electrical stimulation signal or a stem cell enhancing agent) may be applied to any brain area having damaged neural tissue in which exogenous stem cells have been transplanted.

Damaged neural tissue may arise from a genetic source, a disease, and/or a trauma. Damaged neural tissue may result from a neruodegenerative disease, such as Parkinson's disease and Alzheimer's disease. In Parkinson's disease, damage neural tissue may be found in the substantia nigra. In Alzheimer's disease, damaged neural tissue may be found in the basal forebrain, particularly the nucleus basalis of meynert, or the hippocampus, specifically the CA1 region. In a condition such as spinal cord injury, it may be desirable to administer a therapy and exogenous stem cells intrathecally at or near the level of the injury. Damaged neural tissue will be readily identifiable to a physician or other persons skilled in the biomedical arts.

One exemplary therapy includes the administration of the growth factor, TGF-alpha, at a dose and rate sufficient to encourage proliferation, differentiation, migration, or integration of an exogenous transplanted stem cell. A suggested rate is in the range from about 0.2 μl/day to about 24 μl/day. A suggested dose is in the range from about 0.1 mg/ml to about 100 mg/ml.

Another exemplary therapy includes the administration of noggin and BMP to a damaged brain region into which exogenous stem cells have been transplanted. Temporally and spatially controlled administration of BMP and noggin may be achieved using a device(s) as described herein or as known in the art. Exogenous noggin may be delivered to the exogenous stem cells to promote neuronal differentiation whereas exogenous BMP may be delivered to promote glial differentiation.

Another exemplary therapy includes applying an electrical signal to promote the expression of a gene product in the area of the damaged tissue at parameters sufficient to encourage the proliferation, differentiation, migration or integration of the exogenous stem cells. For example, the expression of c-fos, neuroD2, nogging, or various other stem cell enhancing agents may be encouraged. Electrical signal parameters may be in the range from, e.g., about 1 Hz to about 150 Hz, about 90 μsec to about 500 μsec, and about 0.1 V to about 10V.

In addition to delivering a stem cell enhancing agent to a CNS region comprising damaged neural tissue, it may be desirable to deliver such agents intraventricularly or intrathecally. Such non-targeted delivery of therapy may broadly encourage the proliferation, differentiation, migration, or integration of the exogenous stem cells.

Brain Regions to which Neurons Project

In an embodiment, therapy is delivered to a CNS region in which neurons are predicted to project. More particularly, therapy may be administered to a region where differentiated neuronal stem cells are expected to project to facilitate the newly developed or existing yet damaged neurons to make the appropriate neuronal connections.

Regions to which neurons are expected to send projections are known to those of skill in the art. For example, neurons of the substantial nigra send projections to the putamen. Accordingly to treat Parkinson's disease, it may be desirable to encourage newly integrated or differentiated neurons or damaged neurons of the substantia nigra to send projections to the putamen. This may be accomplished by delivering electrical signal, a stem cell enhancing agent, or a combination thereof to the putamen to encourage the neurons of the substantia nigra to make appropriate connections with neurons of the putamen.

In another example, a group of cholinergic neurons in the basal forebrain project to the neocortical and medial temporal regions. In Alzhiemer's disease this group of cholinergic neurons are selectively damaged, resulting in severe impairment of learning. It may be desirable to encourage newly integrated or differentiated neurons of the basal forebrain to send projections to the neocortical and medial temporal regions. Furthermore, it may be desirable to encourage the newly established neuronal cell to produce acetylcholine to restore the function of the cholinergic transmission in the brain area. This may be accomplished by delivering electrical signal, a stem cell enhancing agent, or a combination thereof to the neocortical and the medial temporal regions to encourage the neurons of the basal forebrain to make appropriate connections with neurons of the neocortical or medial temporal region. Likewise, replacement strategy may be achieved by delivering electrical signal, a stem cell enhancing agent, or a combination thereof to the basal forebrain to encourage the neurons of the neocortical and medial temporal regions to make appropriate connections with neurons of the basal forebrain.

Other neurotransmitter systems are selectively disrupted by the Alzheiemer's disease process in a manner similar to the cholinergic system. In another example, the cortically projecting norepinephrine neurons of the locus coeruleus and the raphe neurons of the dorsal and central raphe nuclei are disrupted. It may be desirable to encourage newly differentiated or integrated or damaged neurons of the locus coeruleus and the raphe nucleus to send projections to the cortex. This may be accomplished by delivering electrical signal, a stem cell enhancing agent, or a combination thereof to the locus coeruleus and the raphe nucleus.

In another example, axons of the neurons in the spinal cord may traverse some distance in the spinal cord on their way to project to a particular spinal cord level. During spinal cord injury, these axonal projections are damaged, resulting in impairment of sensory and movement functions and often paralysis. It may be desirable to encourage the newly integrated or differentiated neurons of one spinal cord level to send projections to the other spinal cord level in a manner that will result in an the repair of axonal projections over the injured area.

Therapy

In various embodiments of the invention, transplanted exogenous stem cells and therapy may be delivered to one or more CNS regions to treat a disease associated with loss of neuronal function. Exogenous stem cells are preferably transplanted into a CNS region comprising damaged nervous tissue. One or more therapies, e.g. electrical signal and stem cell enhancing agent, may be delivered to, e.g., the damaged brain region into which the exogenous cells are transplanted or a region to which neurons from the damaged CNS region are predicted to project. In various embodiments, a stem cell enhancing agent and an electrical signal are delivered to the brain region into which the exogenous stem cells are transplanted. If the exogenous stem cell comprises an electrically responsive genetic construct an electrical signal is preferably delivered to the brain region into which the exogenous cells are transplanted.

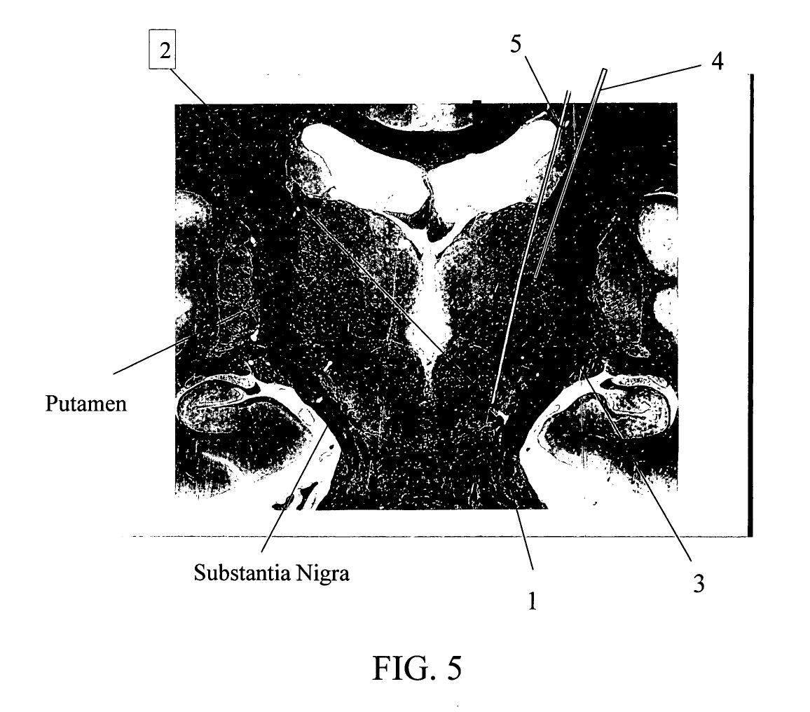

Referring to FIG. 5, an exemplary embodiment useful for treating Parkinson's disease is shown. Exogenous stem cells, which may or may not contain an electrically responsive genetic construct, are implanted into the substantia nigra at step 1. A first therapy delivery element 5 having a therapy delivery region is implanted into the brain of the subject such that the therapy delivery region is positioned in or near the substantia nigra. At step 2, therapy is applied to the substantia nigra to promote proliferation, differentiation, migration, or integration of the exogenous stem cell. A second therapy delivery element having a delivery region is implanted such that the delivery region is positioned in or near the putamen. At step 3, therapy is delivered to the putamen to encourage projections of neurons from the substantia nigra to the putamen. The projections may be from existing neurons or from neurons derived from the exogenous stem cells. The first and second therapy elements may be catheters, leads, or elements comprising both infusion sections and electrodes, or combinations thereof.

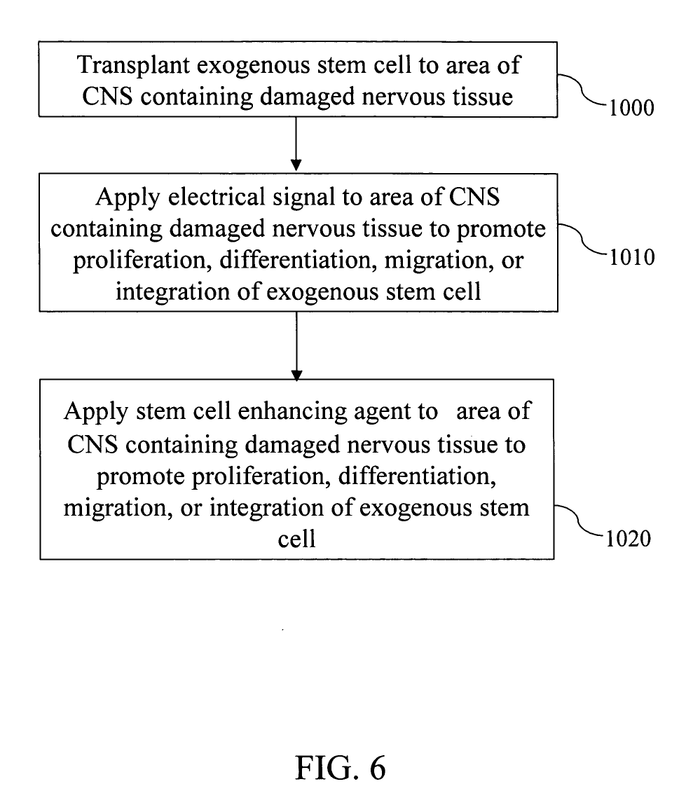

Referring to FIG. 6, an overview of a method of treating a disease associated with a loss of neuronal function is shown. As shown in FIG. 6, an exogenous stem cell is transplanted into an area of the CNS (1000). At 1010, an electrical signal is applied to the area of the CNS into which the exogenous cell was implanted. At 1020, a stem cell enhancing agent is applied to the region into which the exogenous cell was implanted. The stem cell enhancing agent may serve to promote proliferation, differentiation, migration, or integration of the exogenous stem cell.

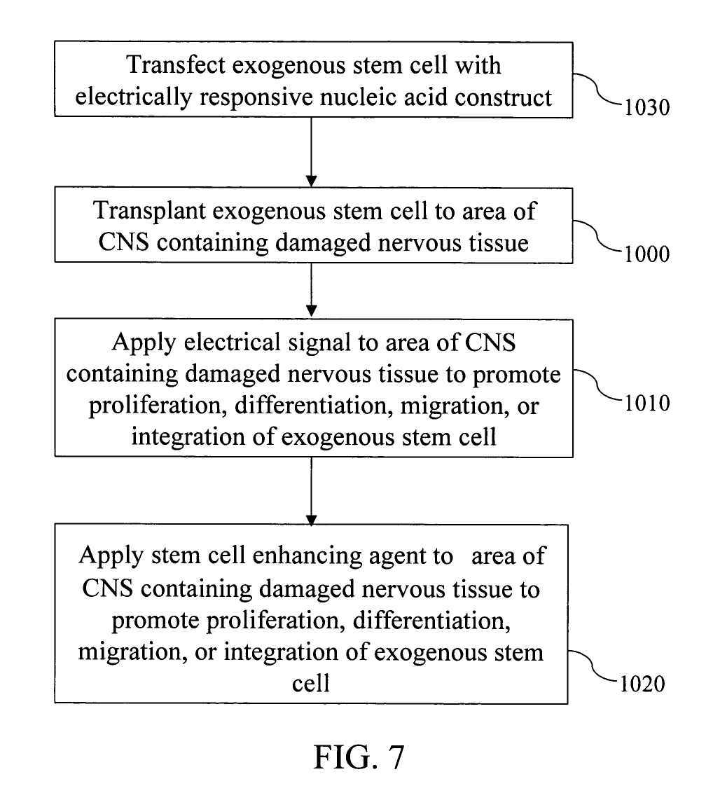

FIG. 7 depicts an overview of a method of treating a disease associated with a loss of neuronal function. The method of depicted in FIG. 7 is similar to that of FIG. 6. In FIG. 7, an additional step of transfecting the endogenous stem cell with an electrically responsive nucleic acid construct is shown at 1030. The electrically responsive nucleic acid construct may comprising a gene encoding a gene product capable of promoting proliferation, differentiation, migration, or integration of the exogenous stem cell. The application of the electrical signal (1010) may control the expression of the gene product.

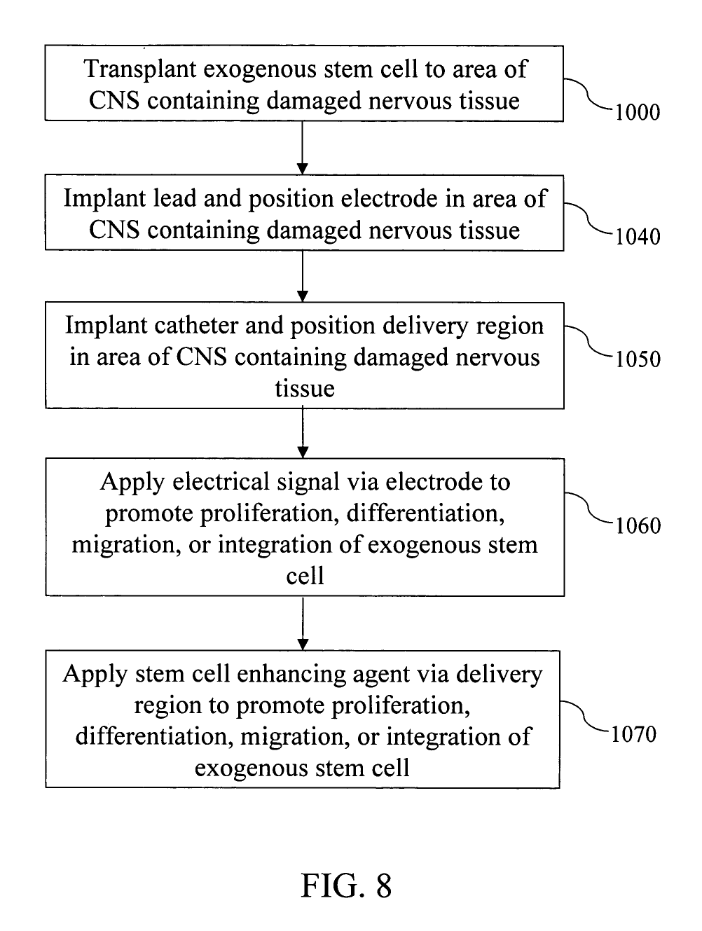

FIG. 8 depicts a method of achieving the treatment protocol described in FIG. 6. An electrode of a lead may be positioned in an area of the brain comprising damaged nervous tissue (1040). Exogenous cells may be transplanted into the damaged CNS region (1000) before of after the lead is implanted and the electrode is positioned (1040). At 1050, a catheter is implanted and a delivery region of the catheter is positioned into an area of the brain comprising damaged nervous tissue (1050). Exogenous cells may be transplanted into the damaged CNS region (1000) before of after the catheter is implanted and the delivery region is positioned (1050). At 1060, an electrical signal is applied via the electrode to promote proliferation, differentiation, migration, or integration of the exogenous stem cell. At 1070, a stem cell enhancing agent is applied via the delivery region to promote proliferation, differentiation, migration, or integration of the exogenous stem cell.

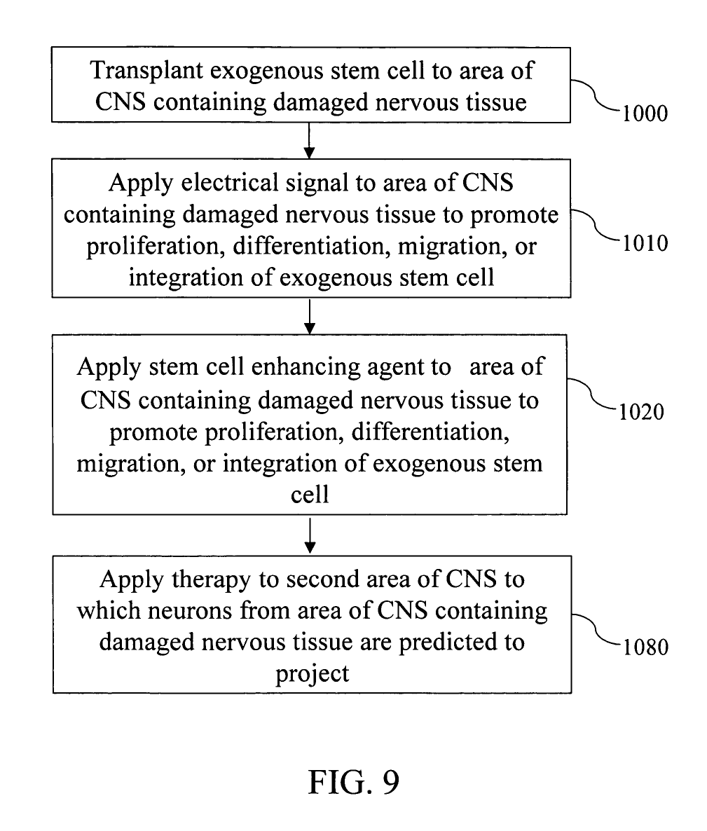

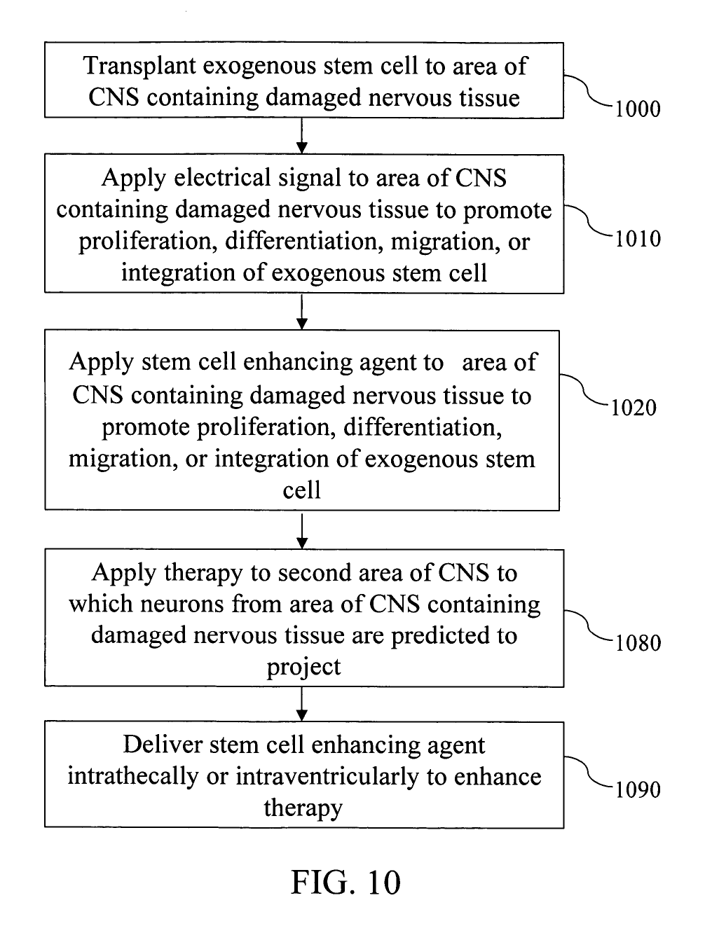

Referring to FIG. 9, an overview of a method of treating a disease associated with a loss of neuronal function is shown. As shown in FIG. 6, an exogenous stem cell is transplanted into an area of the CNS (1000). At 1010, an electrical signal is applied to the area of the CNS into which the exogenous cell was implanted. At 1020, a stem cell enhancing agent is applied to the region into which the exogenous cell was implanted. The stem cell enhancing agent may serve to promote proliferation, differentiation, migration, or integration of the exogenous stem cell. At 1080, a therapy (i.e., an electrical signal or a stem cell enhancing agent) is delivered to a second region of the CNS to which neurons from the damaged CNS region are predicted to project. FIG. 10 shows the additional step of delivering a stem cell enhancing agent intrathecally or intraventricularly to enhance the therapy (1090).

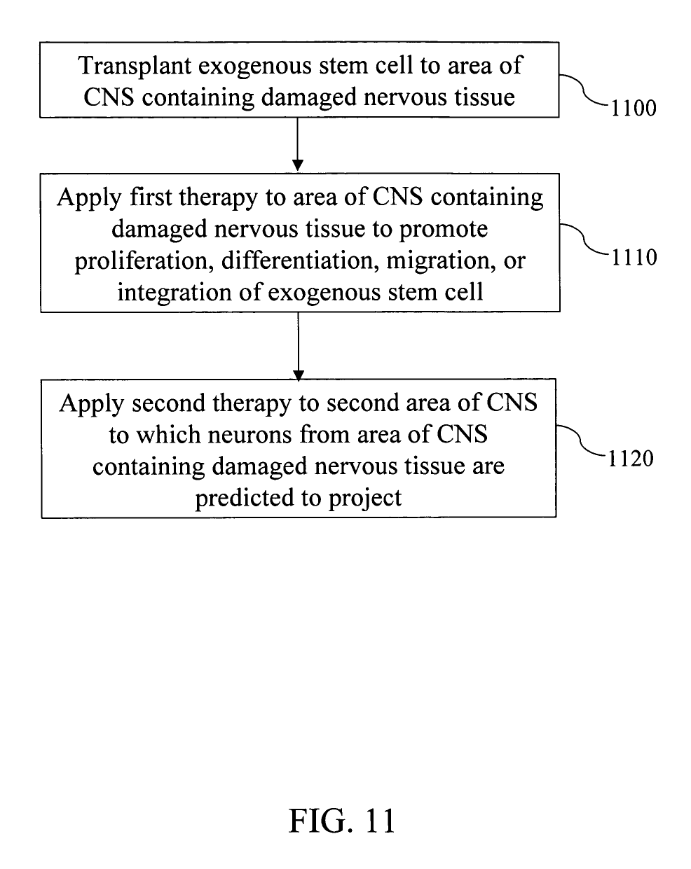

FIG. 11 depicts an overview of a method for treating a disease associated with loss of neuronal function. At 1100, an exogenous stem cell is transplanted to an area of the CNS comprising damaged nervous tissue. At 1100, a first therapy is applied to the area of the CNS comprising damaged nervous tissue. The therapy may serve to promote the proliferation, differentiation, migration, or differentiation of the exogenous stem cell. At 1120, a second therapy is applied to a second CNS region to which neurons from the damaged CNS region are predicted to project. The therapy may serve to promote the projections to ensure proper connections are made. FIG. 122 shows an additional step of delivering a stem cell enhancing agent intrathecally or intraventricularly to enhance therapy (1130).

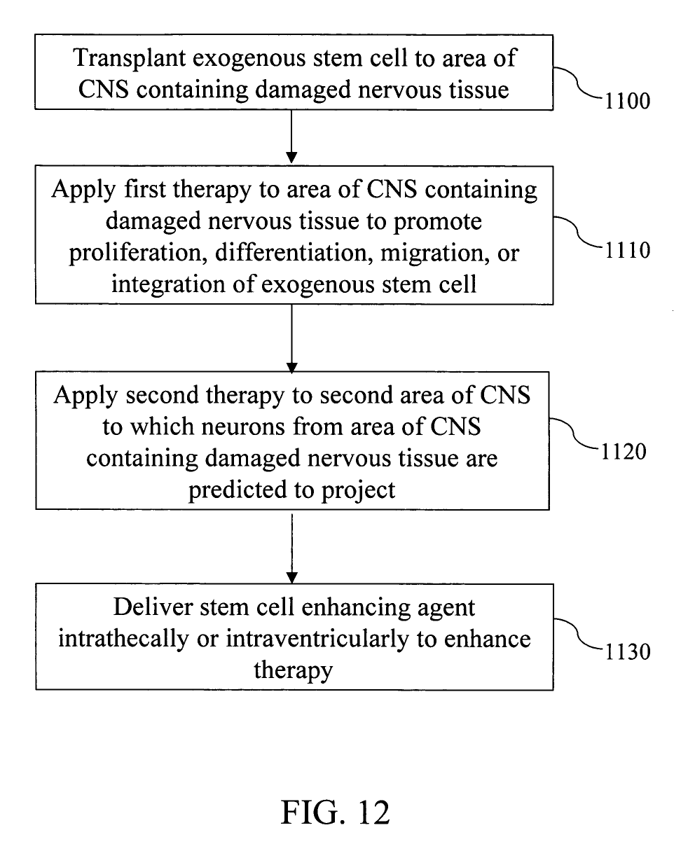

Other methods and combinations of steps shown in FIGS. 6-12 are contemplated. It will be understood that various steps as shown in FIGS. 6-12 may occur in any logical order and applications of various therapies can occur at the same or different times.

All printed publications, such as patents, patent applications, technical papers, and brochures, cited herein are hereby incorporated by reference herein, each in its respective entirety. As those of ordinary skill in the art will readily appreciate upon reading the description herein, at least some of the devices and methods disclosed in the patents and publications cited herein may be modified advantageously in accordance with the teachings of the present invention.

1. Therapeutic delivery system comprising:
a housing;
a electrical signal generator disposed in the housing;
genetically engineered stem cells comprising a target gene operably coupled to an electrically responsive promoter, the cells being operably coupled with the electrical signal generator;
a pump disposed in the housing;
a reservoir operably coupled to the pump; and
one or more stem cell enhancing agents disposed in the reservoir, the one or more stem cell enhancing agents configured to promote the proliferation, migration, differentiation, or integration of a stem cell.

2. The device of claim 1, wherein at least one of the one or more stem cell enhancing agents is selected from the group consisting of GDNF, BDNF, FGF, VEGF, NT-3, TGF-alpha, EGF, IGF-1, CNTF, a glutamate receptor agonist, a GABA receptor antagonist, and an anti-Nogo-A antiboby.

3. The system of claim 1, further comprising:
a lead operably coupled to the pulse generator; and
a catheter operably coupled to the pump.

4. The system of claim 1, wherein the target gene encodes a gene product that promotes the proliferation, differentiation, migration, or integration of the genetically altered stem cell.

5. The system of claim 4, wherein the target gene encodes CNTF, GDNF, BDNF, FGF, VEGF, NT-3, TGF-alpha, TGF-beta, EGF, IGF-1, NT-4, NT-5, EGF, CNTF, SCF, c-fos, NeuroD2, pax6, emx2, SHH, noggin, IL-3 FL, PDGF, FL, Tpo, IL-6, IL-11, or an active derivative or fragment thereof.

6. A method for treating a disease associated with a loss of neuronal function in a subject in need thereof, the method comprising:
transplanting an exogenous stem cell to a first CNS region, the first CNS region comprising damaged neuronal tissue;
implanting a lead in the subject such that an electrode of the lead is positioned in the first CNS region;
implanting a catheter in the subject such that a delivery region of the catheter is positioned in the first CNS region;
applying an electrical signal to first CNS region to promote proliferation, differentiation, migration, or integration of the exogenous stem cell; and
delivering a first stem cell enhancing agent to the first CNS region to promote proliferation, differentiation, migration, or integration of the exogenous stem cell.

7. The method of claim 6, wherein the exogenous stem cell comprises an electrically responsive nucleic acid construct, the construct comprising an electrically responsive promoter and a target gene encoding a gene product capable of promoting the proliferation, differentiation, migration, or proliferation of the exogenous stem cell.

8. The method of claim 7, wherein the applying the electrical signal to first CNS region comprises applying an electrical signal to the first CNS region to induce expression of the target gene product.

9. The method of claim 7, wherein the target gene product is selected from the group consisting of a CNTF, GDNF, BDNF, FGF, VEGF, NT-3, TGF-alpha, TGF-beta, EGF, IGF-1, NT-4, NT-5, EGF, CNTF, SCF, c-fos, NeuroD2, pax6, emx2, SHH, noggin, IL-3 FL, PDGF, FL, Tpo, IL-6 and IL-1.

10. The method of claim 7, wherein the target gene product is an active fragment or derivative of CNTF, GDNF, BDNF, FGF, VEGF, NT-3, TGF-alpha, TGF-beta, EGF, IGF-1, NT-4, NT-5, EGF, CNTF, SCF, c-fos, NeuroD2, pax6, emx2, SHH, noggin, IL-3 FL, PDGF, FL, Tpo, IL-6 or IL-11.

11. The method of claim 6, wherein the stem cell enhancing agent is selected from the group consisting of a growth factor, a chemoattractant, a neurotransmitter receptor agonist or antagonist, a transcription factor, and an inhibitor of a growth inhibitory molecule.

12. The method of claim 11, wherein the growth factor is selected from the group consisting of CNTF, GDNF, BDNF, FGF, VEGF, NT-3, TGF-alpha, EGF, IGF-1, NT-4, NT-5, EGF, CNTF, and SCF.

13. The method of claim 11, wherein the chemoattractant is selected from the group consisting of SDF-1, fractalkine, GRO-α, IL-8, MIP-1a, MIP-1b, MCP-1, MCP-2, MCP-3, GRO-a, GRO-b, GRO-g, RANTES, and eotaxin.

14. The method of claim 11, wherein the neurotransmitter receptor agonist is a glutamate receptor agonist, an alpha 1-adrenergic agonist, an alpha 2-adrenergic agonist, a serotonergic agonist, a dopaminergic agonist, or a GABAergic agonist.

15. The method of claim 11, wherein the neurotransmitter receptor antagonist is a GABA receptor antagonist an alpha 1-adrenergic antagonist, an alpha 2-adrenergic antagonist, a serotonergic antagonist, a dopaminergic antagonist, or a GABAergic antagonist.

16. The method of claim 11, wherein the transcription factor is selected fromteh group consisting of Pax6, EMX2, SHH, a member of the NeuroD family, a member of the CREB family, c-fos, myocyte enhancer factor-2 (MEF-2) and basic helix-loop-helix (bHLH) transcription factors.

17. The method of claim 11, wherein the inhibitor of a growth inhibitory molecule is an inhibitor of amino NogoR receptor signal transduction, a Rho signal transduction inhibitor, and Arginase I.

18. The method of claim 6, wherein the disease is selected from the group consisting of Parkinson's disease, Alzheimer's disease, spinal cord injury, traumatic brain injury, and stroke.

19. The method of claim 18, wherein the disease is Parkinson's disease and the first CNS region is the substantia nigra.

20. The method of claim 18, wherein the disease is Alzheimer's disease and the first CNS region is the forebrain, nucleus basalis of Meynert, neocortical region, medial temporal region, locus ceoruleus, or raphe nucleus.

21. The method of claim 18 wherein the disease is spinal cord injury and the first CNS region is intrathecal at the level of the injury.

22. The method of claim 6, further comprising:
implanting a therapy delivery element comprising a therapy delivery region in the subject and positioning the therapy delivery region of the therapy delivery element in a second CNS region to which neurons from the first CNS region are predicted to project; and
applying a therapy to the second CNS region via the therapy delivery region to promote projections of the neurons from the first CNS region to the second CNS region.

23. The method of claim 22, wherein the projections comprise projections of neurons derived from the exogenous stem cell.

24. The method of claim 22, wherein the projections comprise projections of neurons other than neurons derived from the exogenous stem cell.

25. The method of claim 22, wherein applying therapy to the second CNS region comprises delivering a stem cell enhancing agent.

26. The method of claim 25, wherein the stem cell enhancing agent is selected from the group consisting of anti-Nogo-A antibody, a p75ntr antagonist, a Rho signal transduction inhibitor, and a nogo-66 receptor antagonist, NGF, GDNF, IGF-1, CNTF, and BDNF.

27. The method of claim 22, wherein the disease is Parkinson's disease and the second CNS region comprises the putamen.

28. The method of claim 27, wherein the third therapy comprises GDNF.

29. The method of claim 22, wherein the disease is Alzheimer's disease and the second CNS region comprises the cortex, basal forebrain or nucleus basalis of meynert.

30. The method of claim 29, wherein the third therapy comprises NGF.

31. The method of claim 22, wherein the disease is spinal cord injury and the second CNS region comprises a spinal location where the injured neurons typically send projections.

32. The method of claim 31, wherein the third therapy comprises a stem cell enhancing agent selected from the group consisting of GDNF, BDNF, and VEGF.

33. The method of claim 6, further comprising intraventricularly or intrathecally delivering a second stem cell enhancing agent, the second stem cell enhancing agent being the same or different than the first stem cell enhancing agent.

34. A method for treating a disease associated with a loss of neuronal function in a subject in need thereof, the method comprising:
transplanting an exogenous stem cell to a first CNS region, the first CNS region comprising damaged neuronal tissue;
implanting a first therapy delivery element comprising a therapy delivery region in the subject and positioning the therapy delivery region of the first therapy delivery element in the first CNS region;
implanting a second therapy delivery element comprising a therapy delivery region in the subject and positioning the therapy delivery region of the second therapy delivery element in a second CNS region to which neurons from the first CNS region are predicted to project;
applying a first therapy to the first CNS region via the therapy delivery region of the first therapy delivery element to promote proliferation, differentiation, migration, or integration of the exogenous stem cell; and
applying a second therapy to the second CNS region via the therapy delivery region of the second therapy delivery element to promote projections of the neurons from the first CNS region to the second CNS region.
wherein the first and second therapy are the same or different.

35. The method of claim 34, wherein the exogenous stem cell comprises an electrically responsive nucleic acid construct, the construct comprising an electrically responsive promoter and a target gene encoding a gene product capable of promoting the proliferation, differentiation, migration, or proliferation of the exogenous stem cell.

36. The method of claim 35, wherein the applying the first therapy to the first CNS region comprises applying an electrical signal to the first CNS region to induce expression of the target gene product.

37. The method of claim 35, wherein the target gene product is selected from the group consisting of a CNTF, GDNF, BDNF, FGF, VEGF, NT-3, TGF-alpha, TGF-beta, EGF, IGF-1, NT-4, NT-5, EGF, CNTF, SCF, c-fos, NeuroD2, pax6, emx2, SHH, noggin, IL-3 FL, PDGF, FL, Tpo, IL-6 and IL-1.

38. The method of claim 35, wherein the target gene product is an active fragment or derivative of CNTF, GDNF, BDNF, FGF, VEGF, NT-3, TGF-alpha, TGF-beta, EGF, IGF-1, NT-4, NT-5, EGF, CNTF, SCF, c-fos, NeuroD2, pax6, emx2, SHH, noggin, IL-3 FL, PDGF, FL, Tpo, IL-6 or IL-1.

39. The method of claim 34, wherein the projections comprise projections of neurons derived from the exogenous stem cell.

40. The method of claim 34, wherein the projections comprise projections of neurons other than neurons derived from the exogenous stem cell.

41. The method of claim 34, further comprising intraventricularly or intrathecally delivering a stem cell enhancing agent to promote the proliferation, differentiation, migration, or integration of the exogenous stem cell or a cell derived therefrom.

42. The method of claim 34, wherein at least one of the first and second therapies comprise a stem cell enhancing agent.

43. The method of claim 42, wherein the stem cell enhancing agent is selected from the group consisting of a growth factor, a chemoattractant, a neurotransmitter receptor agonist or antagonist, and an inhibitor of a growth inhibitory molecule.

44. The method of claim 43, wherein the growth factor is selected from the group consisting of CNTF, GDNF, BDNF, FGF, VEGF, NT-3, TGF-alpha, EGF, IGF-1, NT-4, NT-5, EGF, CNTF, and SCF.

45. The method of claim 43, wherein the chemoattractant is selected from the group consisting of SDF-1, fractalkine, GRO-a, IL-8, MIP-1a, MIP-1b, MCP-1, MCP-2, MCP-3, GRO-a, GRO-b, GRO-g, RANTES, and eotaxin.

46. The method of claim 43, wherein the neurotransmitter receptor agonist is a glutamate receptor agonist, an alpha 1-adrenergic agonist, an alpha 2-adrenergic agonist, a serotonergic agonist, a dopaminergic agonist, or a GABAergic agonist.

47. The method of claim 43, wherein the neurotransmitter receptor antagonist is a GABA receptor antagonist an alpha 1-adrenergic antagonist, an alpha 2-adrenergic antagonist, a serotonergic antagonist, a dopaminergic antagonist, or a GABAergic antagonist.

48. The method of claim 43, wherein the inhibitor of a growth inhibitory molecule is anti-Nogo-A antibody, a p75ntr antagonist, a Rho signal transduction inhibitor, and a nogo-66 receptor antagonist.

49. The method of claim 34, wherein the disease is selected from the group consisting of Parkinson's disease, Alzheimer's disease, spinal cord injury, traumatic brain injury, and stroke.

50. The method of claim 49, wherein the disease is Parkinson's disease and the first CNS region is the substantia nigra.

51. The method of claim 49, wherein the disease is Alzheimer's disease and the first CNS region is the forebrain, nucleus basalis of Meynert, neocortical region, medial temporal region, locus ceoruleus, or raphe nucleus.

52. The method of claim 49, wherein the disease is spinal cord injury and the first CNS region is intrathecal at the level of the injury.

53. The method of claim 34, wherein the applying the second therapy to the second CNS region comprises delivering a stem cell enhancing agent to the second CNS region.

54. The method of claim 53, wherein the stem cell enhancing agent is selected from the group consisting of anti-Nogo-A antibody, a p75ntr antagonist, a Rho signal transduction inhibitor, and a nogo-66 receptor antagonist, NGF, GDNF, IGF-1, CNTF, and BDNF.

55. The method of claim 34, wherein the disease is Parkinson's disease and the second CNS region comprises the putamen.

56. The method of claim 55, wherein the second therapy comprises GDNF.

57. The method of claim 34, wherein the disease is Alzheimer's disease and the second CNS region comprises the cortex, basal forebrain or nucleus basalis of meynert.

58. The method of claim 57, wherein the second therapy comprises NGF.

59. The method of claim 34, wherein the disease is spinal cord injury and the second CNS region comprises a spinal location where the injured neurons typically send projections.

60. The method of claim 59, wherein the third therapy comprises a stem cell enhancing agent selected from the group consisting of GDNF, BDNF, and VEGF.
